(19) United States
(12) Patent Application Publication
(10) Pub. No.: US 2004/0186389 A1
(43) Pub. Date: Sep. 23, 2004

(54) Apparatus and method for determining a respiratory quotient

(76) Inventors: James R. Mault, Evergreen, CO (US); Edwin M. Pearce, Golden, CO (US); Thomas E. Kilbourn, Saratoga, CA (US); Geoffrey G. Moyer, Portola Valley, CA (US)

Correspondence Address: COOLEY GODWARD, LLP, 3000 EL CAMINO REAL, 5 PALO ALTO SQUARE, PALO ALTO, CA, 94306, US

(21) Appl. No.: 10/672,071

(22) Filed: Sep. 25, 2003

Related U.S. Application Data
(63) Continuation-in-part of application No. 10/128,105, filed on Apr. 23, 2002, now Pat. No. 6,645,158.
Application No. 10/128,105 is a continuation of application No. 09/601,589, filed on Sep. 19, 2000, now Pat. No. 6,402,698.
Application No. 09/601,589 is a 371 of international application No. PCT/US99/02448, filed on Feb. 5, 1999.
(60) Provisional application No. 60/413,505, filed on Sep. 25, 2002.
(60) Provisional application No. 60/073,812, filed on Feb. 5, 1998.
(60) Provisional application No. 60/104,983, filed on Oct. 20, 1998.

(51) Int. Cl. A61B 5/08

(52) U.S. Cl. 600/531

(57) ABSTRACT

Apparatus and methods for determining a respiratory quotient are described. In one embodiment, a respiratory gas exchange monitor includes a respiratory gas conduit, a respiratory gas flow meter coupled to the respiratory gas conduit, and a respiratory gas sensor coupled to the respiratory gas conduit. The respiratory gas exchange monitor also includes a computation unit coupled to the respiratory gas flow meter and the respiratory gas sensor. The computation unit is configured to process outputs of the respiratory gas flow meter and the respiratory gas sensor to determine an amount of carbon dioxide produced by a subject and an amount of oxygen consumed by the subject, and the computation unit is configured to determine a respiratory quotient of the subject based on the amount of carbon dioxide produced and the amount of oxygen consumed.

### CROSS REFERENCE TO RELATED APPLICATIONS

This application claims the benefit of U.S. Provisional Application No. 60/413,505, filed on Sep. 25, 2002, and is a continuation-in-part of U.S. patent application Ser. No. 10/128,105, filed on Apr. 23, 2002, which is a continuation of U.S. patent application Ser. No. 09/601,589, which is the National Stage of International Application No. PCT/US99/02448, filed on Feb. 5, 1999, which claims the benefit of U.S. Provisional Application No. 60/073,812, filed on Feb. 5, 1998, and U.S. Provisional Application No. 60/104,983, filed on Oct. 20, 1998, the disclosures of which are incorporated herein by reference in their entirety.

### BRIEF DESCRIPTION OF THE INVENTION

The invention relates generally to analyzing respiratory gases. More particularly, the invention relates to apparatus and methods for analyzing respiratory gases of a subject to determine a respiratory quotient of the subject.

### BACKGROUND OF THE INVENTION

Cellular respiration refers to biological processes associated with oxidative metabolism in cells. Cellular respiration typically involves the consumption of oxygen and the production of carbon dioxide, water, and adenosine triphosphate during oxidation of a metabolic substrate. Examples of metabolic substrates include carbohydrates, lipids, and proteins.

The amount of oxygen consumed and the amount of carbon dioxide produced during oxidation of a particular metabolic substrate can be determined. For example, oxidation of a molecule of glucose typically involves the following relationship: 6O₂+C₆H₁₂O₆=>6CO₂+6H₂O+38 ATP. Thus, when glucose is used as a metabolic substrate, the number of oxygen molecules consumed is typically equal to the number of carbon dioxide molecules produced. The ratio of the amount of carbon dioxide produced and the amount of oxygen consumed is typically referred to as a respiratory quotient. As discussed above, the respiratory quotient is typically 1.0 when glucose is used as a metabolic substrate. The respiratory quotient is also typically 1 when other types of carbohydrates are used as metabolic substrates. The respiratory quotient is typically 0.71 for oxidation of lipids and 0.82 for oxidation of proteins. For oxidation of a mixture of carbohydrates, lipids, and proteins, the respiratory quotient is typically in the range of 0.80 to 0.85.

Respiratory quotients are sometimes estimated to determine other physiological parameters, such as, for example, metabolic rates. However, determination of an “actual” respiratory quotient can yield valuable information about aggregate metabolic processes of an individual. For example, determination of an “actual” respiratory quotient of the individual can yield information relating to the individual's nutritional status. Such information can find use in nutritional therapy and can ensure that nutritional requirements of the individual are met.

Various devices are available for analyzing respiratory gases. However, existing devices sometimes do not include functionality to determine an “actual” respiratory quotient of an individual. Other existing devices are relatively bulky, expensive, and difficult to operate. For example, to determine the amount of oxygen in respiratory gases, some existing devices require the use of a scrubber that removes carbon dioxide from the respiratory gases. Also, some existing devices require the use of multiple respiratory gas sensors to determine the amounts of different components of respiratory gases. For example, to determine the amount of carbon dioxide and the amount of oxygen in respiratory gases, some existing devices require the use of one respiratory gas sensor for carbon dioxide and another respiratory gas sensor for oxygen.

It is against this background that a need arose to develop the apparatus and methods described herein.

### SUMMARY OF THE INVENTION

In one embodiment, a respiratory gas exchange monitor includes a respiratory gas conduit configured to convey inhaled gases and exhaled gases of a subject. The respiratory gas exchange monitor also includes a respiratory gas flow meter coupled to the respiratory gas conduit, and the respiratory gas flow meter is configured to generate an output associated with a volume of the inhaled gases and a volume of the exhaled gases. The respiratory gas exchange monitor also includes a respiratory gas sensor coupled to the respiratory gas conduit, and the respiratory gas sensor is configured to generate an output associated with a concentration of oxygen in the exhaled gases. The respiratory gas exchange monitor further includes a computation unit coupled to the respiratory gas flow meter and the respiratory gas sensor. The computation unit is configured to process the output of the respiratory gas flow meter and the output of the respiratory gas sensor to determine an amount of carbon dioxide produced by the subject and an amount of oxygen consumed by the subject, and the computation unit is configured to determine a respiratory quotient of the subject based on the amount of carbon dioxide produced and the amount of oxygen consumed.

In another embodiment, a respiratory gas exchange monitor includes a respiratory gas flow meter configured to generate an output associated with inhaled gases and exhaled gases of a subject. The respiratory gas exchange monitor also includes a respiratory gas sensor configured to generate an output associated with the exhaled gases. The respiratory gas exchange monitor further includes a computation unit coupled to the respiratory gas flow meter and the respiratory gas sensor. The computation unit is configured to process the output of the respiratory gas flow meter to determine a volume of the inhaled gases and a volume of the exhaled gases, and the computation unit is configured to process the output of the respiratory gas sensor to determine a concentration of oxygen in the exhaled gases. The computation unit is configured to determine an amount of carbon dioxide produced by the subject and an amount of oxygen consumed by the subject based on the volume of the inhaled gases, the volume of the exhaled gases, and the concentration of oxygen in the exhaled gases, and the computation unit is configured to determine a respiratory quotient of the subject based on a ratio of the amount of carbon dioxide produced and the amount of oxygen consumed.

In another embodiment, a respiratory gas exchange monitor includes a respiratory gas flow meter configured to generate an output associated with inhaled gases and exhaled gases of a subject. The respiratory gas exchange monitor also includes a computation unit coupled to the respiratory gas flow meter. The computation unit is configured to process the output of the respiratory gas flow meter to determine a volume of the inhaled gases, a volume of the exhaled gases, and a mass of the exhaled gases. The computation unit is configured to determine an amount of carbon dioxide produced by the subject and an amount of oxygen consumed by the subject based on the volume of the inhaled gases, the volume of the exhaled gases, and the mass of the exhaled gases, and the computation unit is configured to determine a respiratory quotient of the subject based on a ratio of the amount of carbon dioxide produced and the amount of oxygen consumed.

In another embodiment, a respiratory gas exchange monitor includes a conduit configured to convey inhaled gases and exhaled gases of a subject. The respiratory gas exchange monitor also includes a first sensor coupled to the conduit, and the first sensor is configured to generate a first signal associated with a volume of the inhaled gases and a volume of the exhaled gases. The respiratory gas exchange monitor also includes a second sensor coupled to the conduit, and the second sensor is configured to generate a second signal associated with a concentration of oxygen in the exhaled gases. The respiratory gas exchange monitor further includes a computation unit coupled to the first sensor and the second sensor. The computation unit is configured to process the first signal and the second signal to determine an amount of carbon dioxide produced by the subject and an amount of oxygen consumed by the subject, and the computation unit is configured to determine a respiratory quotient of the subject based on the amount of carbon dioxide produced and the amount of oxygen consumed.

In another embodiment, a respiratory gas exchange monitor includes means for determining a volume of inhaled gases of a subject and a volume of exhaled gases of the subject. The respiratory gas exchange monitor also includes means for determining a concentration of oxygen in the exhaled gases. The respiratory gas exchange monitor also includes means for determining an amount of carbon dioxide produced by the subject and an amount of oxygen consumed by the subject based on the volume of said inhaled gases, the volume of the exhaled gases, and the concentration of oxygen in the exhaled gases. The respiratory gas exchange monitor further includes means for determining a respiratory quotient of the subject based on a ratio of the amount of carbon dioxide produced and the amount of oxygen consumed.

In another embodiment, a respiratory gas exchange monitor is configured to perform a method that includes determining a volume of inhaled gases and a volume of exhaled gases. The method also includes determining a speed of sound in the exhaled gases. The method also includes determining an amount of carbon dioxide produced and an amount of oxygen consumed based on the volume of the inhaled gases, the volume of the exhaled gases, and the speed of sound in the exhaled gases. The method further includes determining a respiratory quotient based on the amount of carbon dioxide produced and the amount of oxygen consumed.

In a further embodiment, a method includes determining a volume of inhaled gases of a subject and a volume of exhaled gases of the subject. The method also includes determining a mass of carbon dioxide and oxygen in the exhaled gases. The method also includes determining a concentration of oxygen in the exhaled gases based on the mass of carbon dioxide and oxygen in the exhaled gases. The method also includes determining an amount of carbon dioxide produced by the subject and an amount of oxygen consumed by the subject based on the volume of the inhaled gases, the volume of the exhaled gases, and the concentration of oxygen in the exhaled gases. The method further includes determining a respiratory quotient of the subject based on the amount of carbon dioxide produced and the amount of oxygen consumed.

Other embodiments and aspects for determining a respiratory quotient of a subject are also contemplated. The foregoing summary and the following detailed description are not meant to restrict the invention disclosed herein to any particular embodiment but are merely meant to describe some embodiments of the invention.

### BRIEF DESCRIPTION OF THE DRAWINGS

For a better understanding of the nature and objects of some embodiments of the invention, reference should be made to the following detailed description taken in conjunction with the accompanying drawings, in which:

FIG. 1 illustrates a respiratory gas exchange monitor in accordance with an embodiment of the invention;

FIG. 2 illustrates a flow chart for determining a respiratory quotient of a subject in accordance with an embodiment of the invention;

FIG. 3 illustrates a flow chart for determining a respiratory quotient of a subject in accordance with another embodiment of the invention; and

FIG. 4 illustrates a respiratory gas exchange monitor in accordance with another embodiment of the invention.

### DETAILED DESCRIPTION

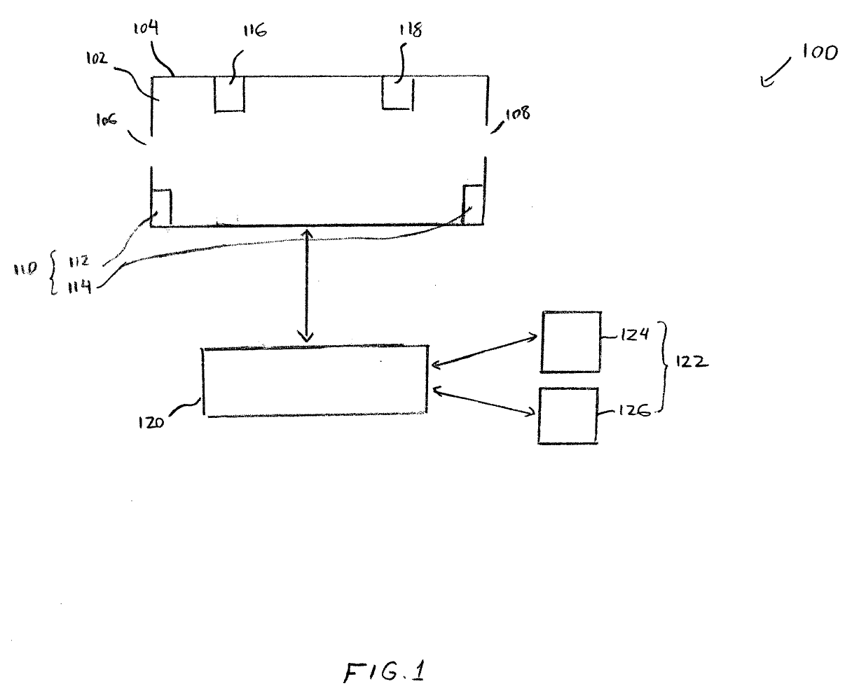

FIG. 1 illustrates a respiratory gas exchange monitor 100 implemented in accordance with an embodiment of the invention. The respiratory gas exchange monitor 100 can be operated to analyze respiratory gases of a subject. In the illustrated embodiment, the respiratory gas exchange monitor 100 can be operated to analyze respiratory gases of the subject to determine a number of respiratory parameters, including a respiratory quotient of the subject. The subject can be any organism for which a respiratory quotient can be determined. For example, the subject can be a warm-blooded animal such as a human.

As illustrated in FIG. 1, the respiratory gas exchange monitor 100 includes a respiratory gas conduit 102, which is configured to convey respiratory gases of the subject. In the illustrated embodiment, the respiratory gas conduit 102 includes a flow tube 104, which includes a pair of openings 106 and 108. The opening 106 is configured to interface with the subject so as to provide inhaled gases to the subject and to receive exhaled gases from the subject. It is contemplated that the opening 106 can interface with the subject via a respiratory gas connector (not illustrated in FIG. 1). For certain applications, the respiratory gas connector can include a mask that can be retained over the subject's face so as to cover the subject's nose and mouth. For other applications, the respiratory gas connector can include a mouthpiece that can be retained over the subject's face so as to cover the subject's mouth, and a nose clip can be used to seal the subject's nostrils.

The opening 108 is configured to interface with ambient air. When the subject inhales, ambient air is drawn into the flow tube 104 via the opening 108 and is conveyed to the subject via the opening 106 as inhaled gases. When the subject exhales, exhaled gases are drawn into the flow tube 104 via the opening 106 and are conveyed into ambient air via the opening 108. Thus, in the illustrated embodiment, inhaled gases and exhaled gases of the subject pass through the flow tube 104 along substantially opposite directions. While not illustrated in FIG. 1, it is contemplated that a flow disruptor can be positioned with respect to the flow tube 104, such that respiratory gases passing through the flow tube 104 are adequately mixed. For certain applications, the flow disruptor can include a protrusion or a baffle to generate turbulence in respiratory gases passing through the flow tube 104.

As illustrated in FIG. 1, the respiratory gas exchange monitor 100 also includes a respiratory gas flow meter or sensor 110, which is coupled to the respiratory gas conduit 102. The respiratory gas flow meter 110 is configured to generate an output associated with respiratory gases passing through the flow tube 104. In particular, the respiratory gas flow meter 110 is configured to generate a set of outputs associated with a volume of inhaled gases passing through the flow tube 104 and a volume of exhaled gases passing through the flow tube 104. In some instances, the set of outputs generated by the respiratory gas flow meter 110 can be in the form of signals, such as, for example, electrical signals. In the illustrated embodiment, the set of outputs generated by the respiratory gas flow meter 110 is associated with flow rates of the inhaled gases and the exhaled gases passing through the flow tube 104 at various times. Because the flow tube 104 has known dimensions, determining flow rates of the inhaled gases and the exhaled gases allows the volume of the inhaled gases and the volume of the exhaled gases to be determined.

In the illustrated embodiment, the respiratory gas flow meter 110 is desirably an ultrasonic flow meter and includes a pair of spaced apart ultrasonic transducers 112 and 114. The ultrasonic transducers 112 and 114 are positioned with respect to the flow tube 104, such that ultrasonic pulses transmitted between the ultrasonic transducers 112 and 114 can travel along a direction that is substantially aligned with respect to a direction along which respiratory gases pass through the flow tube 104. While this positioning of the ultrasonic transducers 112 and 114 can be desirable to improve measurement accuracy, it is contemplated that the positioning of the ultrasonic transducers 112 and 114 can be varied from that illustrated in FIG. 1. For example, the ultrasonic transducers 112 and 114 can be positioned such that ultrasonic pulses travel along a direction that forms any angle with respect to a direction along which respiratory gases pass through the flow tube 104. Also, while not illustrated in FIG. 1, it is contemplated that anti-microbial filters can be used to cover exposed surfaces of the ultrasonic transducers 112 and 114.

Using the ultrasonic transducers 112 and 114, ultrasonic pulses can be transmitted with and against a direction along which respiratory gases pass through the flow tube 104, resulting in a set of outputs associated with “upstream” and “downstream” transit times. When a flow rate of respiratory gases is zero, “upstream” and “downstream” transit times are typically the same. However, when a flow rate of respiratory gases is not zero, “upstream” and “downstream” transit times can differ, and the difference between the “upstream” and “downstream” transit times is dependent on the flow rate. For certain applications, ultrasonic pulses can be transmitted in an alternating fashion with and against a direction along which respiratory gases pass through the flow tube 104.

The ultrasonic transducers 112 and 114 can be implemented as, for example, described in U.S. Pat. Nos. 5,419,326; 5,503,151; 5,562,101; 5,645,071; 5,647,370; 5,777,238; 5,831,175; 6,189,389; and PCT Publication No. WO 00/07498; the disclosures of which are incorporated herein by reference in their entirety. The ultrasonic transducers 112 and 114 can also be implemented as, for example, described in K. K. Shung (Ed.), Ultrasonic Transducer Engineering (Medical Imaging 1999), Society of Photo-optical Instrumentation Engineers (ISBN: 081943162), the disclosure of which is incorporated herein by reference in its entirety. In addition, the ultrasonic transducers 112 and 114 can be implemented using piezoelectric crystal transducers as, for example, described in U.S. Pat. Nos. 2,911,825; 5,214,966; and 6,277,645; the disclosures of which are incorporated herein by reference in their entirety. Such piezoelectric crystal transducers can generate a set of outputs associated with flow rates as well as densities of respiratory gases at various times. For certain applications, the ultrasonic transducers 112 and 114 can be implemented using commercially available ultrasonic transducers, such as, for example, those available from SECO Sensor Consult GmbH, Coburg, Germany. Other types of ultrasonic flow meters can be used, such as, for example, micromachined ultrasonic transducer arrays and ultrasonic flow meters using a sing-around method of determining flow rates. Also, other approaches of determining flow rates can be used in place of, or in conjunction with, an ultrasonic flow meter. For example, flow rates can be determined using differential pressure flow meters, mass flow meters, rotating vane flow meters, and thermal convection flow meters.

As illustrated in FIG. 1, the respiratory gas exchange monitor 100 also includes a respiratory gas sensor 116, which is coupled to the respiratory gas conduit 102. The respiratory gas sensor 116 is configured to generate an output associated with respiratory gases passing through the flow tube 104. In particular, the respiratory gas sensor 116 is configured to generate a set of outputs associated with a composition of inhaled gases passing through the flow tube 104, a composition of exhaled gases passing through the flow tube 104, or both. In some instances, the set of outputs generated by the respiratory gas sensor 1 16 can be in the form of signals, such as, for example, electrical signals. In the illustrated embodiment, the set of outputs generated by the respiratory gas sensor 116 is associated with concentrations of oxygen in respiratory gases passing through the flow tube 104 at various times. Because a volume of respiratory gases passing through the flow tube 104 can be determined, determining concentrations of oxygen in the respiratory gases allows an amount of oxygen in the respiratory gases to be determined. In addition, determining concentrations of oxygen in the respiratory gases allows an amount of carbon dioxide in the respiratory gases to be determined.

In the illustrated embodiment, the respiratory gas sensor 116 is desirably an oxygen sensor, such as, for example, a fluorescence quench oxygen sensor. The respiratory gas sensor 116 is positioned with respect to the flow tube 104, such that respiratory gases passing through the flow tube 104 are in contact with the respiratory gas sensor 116. Advantageously, the respiratory gas sensor 116 is positioned so as to expose it to a turbulent flow of respiratory gases. While this positioning of the respiratory gas sensor 116 can be desirable to improve measurement accuracy, it is contemplated that the positioning of the respiratory gas sensor 116 can be varied from that illustrated in FIG. 1. Also, while not illustrated in FIG. 1, it is contemplated that an anti-microbial filter can be used to cover an exposed surface of the respiratory gas sensor 116.

Using the respiratory gas sensor 116, a fluorescent material included in the respiratory gas sensor 116 can be intermittently excited with an incident radiation, resulting in a set of outputs associated with fluorescence intensities of the fluorescent material. Concentrations of oxygen at various times can be determined based on a decrease in the fluorescence intensities as a result of oxygen quenching. When a lesser amount of oxygen is present in respiratory gases, a greater fluorescence intensity can be detected. However, when a greater amount of oxygen is present in the respiratory gases, a lesser fluorescence intensity can be detected.

The respiratory gas sensor 116 can be implemented as, for example, described in U.S. Pat. Nos. 5,517,313; 5,894,351; 5,910,661; 5,917,605; and PCT Publication No. WO 00/13003; the disclosures of which are incorporated herein by reference in their entirety. For certain applications, the respiratory gas sensor 116 can be implemented using commercially available oxygen sensors, such as, for example, those available from Sensors for Medicine and Science, Inc., Germantown, Md. Other types of oxygen sensors can be used, such as, for example, paramagnetic oxygen sensors and polarographic oxygen sensors. Also, other approaches of determining a composition of respiratory gases can be used in place of, or in conjunction with, an oxygen sensor. In particular, the respiratory gas sensor 116 can be configured to generate a set of outputs associated with concentrations of other components of respiratory gases passing through the flow tube 104. For example, the respiratory gas sensor 116 can be a carbon dioxide sensor and can be configured to generate a set of outputs associated with concentrations of carbon dioxide in the respiratory gases at various times.

As illustrated in FIG. 1, the respiratory gas exchange monitor 100 also includes an environmental sensor 118, which is coupled to the respiratory gas conduit 102. The environmental sensor 118 is configured to generate an output associated with respiratory gases passing through the flow tube 104. In particular, the environmental sensor 118 is configured to generate a set of outputs associated with various respiratory parameters of inhaled gases passing through the flow tube 104, various respiratory parameters of exhaled gases passing through the flow tube 104, or both. In some instances, the set of outputs generated by the environmental sensor 118 can be in the form of signals, such as, for example, electrical signals. In the illustrated embodiment, the set of outputs generated by the environmental sensor 118 is associated with respiratory parameters such as, for example, pressure, relative humidity, and temperature. The environmental sensor 118 is positioned with respect to the flow tube 104, such that respiratory gases passing through the flow tube 104 are in contact with the environmental sensor 118. While this positioning of the environmental sensor 118 can be desirable to improve measurement accuracy, it is contemplated that the positioning of the environmental sensor 118 can be varied from that illustrated in FIG. 1. Also, while not illustrated in FIG. 1, it is contemplated that an anti-microbial filter can be used to cover an exposed surface of the environmental sensor 118.

The environmental sensor 118 can be implemented using a pressure sensor, a relative humidity sensor, and a temperature sensor. For certain applications, the environmental sensor 118 can be implemented using commercially available sensors for determining pressure, relative humidity, and temperature, such as, for example, temperature sensors available from Thermometrics, Edison, N. J., pressure sensors available from Motorola, Inc., Schaumburg, Ill., and relative humidity sensors available from Honeywell International Inc., Morristown, N.J. Other types of environmental sensors can be used, such as, for example, an integrated sensor for determining pressure, relative humidity, and temperature.

As illustrated in FIG. 1, the respiratory gas exchange monitor 100 also includes a computation unit 120, which is coupled to the respiratory gas flow meter 110, the respiratory gas sensor 116, and the environmental sensor 118. In particular, the computation unit 120 is electronically coupled to the respiratory gas flow meter 110, the respiratory gas sensor 116, and the environmental sensor 118 via any wire or wireless transmission channel. The computation unit 120 is configured to control operation of one or more of the respiratory gas flow meter 110, the respiratory gas sensor 116, and the environmental sensor 118. In addition, the computation unit 120 is configured to process outputs of the respiratory gas flow meter 110, the respiratory gas sensor 116, and the environmental sensor 118 to determine an amount of carbon dioxide produced by the subject and an amount of oxygen consumed by the subject. The computation unit 120 is configured to determine a respiratory quotient of the subject based on a ratio of the amount of carbon dioxide produced and the amount of oxygen consumed.

In the illustrated embodiment, the computation unit 120 is configured to compare a respiratory quotient of the subject with a reference respiratory quotient, such as, for example, an expected or a target respiratory quotient, and the computation unit 120 is configured to determine a measure of deviation of the respiratory quotient with respect to the reference respiratory quotient. The reference respiratory quotient can be specified by a user or can be determined by the computation unit 120 based on physiological parameters of the subject, such as, for example, blood glucose level, blood pressure, heart rate, metabolic rate, nutritional intake, physical activity, and pulmonary function.

The computation unit 120 can be implemented using one or more of the following: (1) a dedicated hardware circuitry, such as, for example, an application specific integrated circuit or a programmable gate array; (2) a computer-readable medium storing computer-executable software code; (3) a conventional microprocessor or central processing unit; and (4) a conventional personal computer, a wireless communication device, or a personal digital assistant. For example, the computation unit 120 can be implemented using a conventional microprocessor that includes a memory, input/output port, an instruction set, and a communication port.

As illustrated in FIG. 1, the respiratory gas exchange monitor 100 further includes a set of input/output devices 122, which is coupled to the computation unit 120. In particular, the set of input/output devices 122 is electronically coupled to the computation unit 120 via any wire or wireless transmission channel. In the illustrated embodiment, the set of input/output devices 122 includes a display device 124 and a data entry device 126. The display device 124 is configured to provide indicia of various respiratory parameters determined by the computation unit 120, such as, for example, a volume of inhaled gases, a volume of exhaled gases, a composition of inhaled gases, a composition of exhaled gases, an amount of carbon dioxide produced, an amount of oxygen consumed, a respiratory quotient of the subject, a reference respiratory quotient, a measure of deviation of the respiratory quotient with respect to the reference respiratory quotient, and so forth. The display device 124 can be implemented using, for example, a display screen and associated hardware circuitry, a computer monitor, a flat panel display, a personal digital assistant, or a wireless communication device.

The data entry device 126 is configured to allow specification of various processing options in connection with determining a respiratory quotient of the subject. For example, the data entry device 126 can be used to specify a start or stop command or various physiological parameters of the subject. The data entry device 126 can be implemented using, for example, a keyboard, a mouse, a pushbutton, or a voice recognition module.

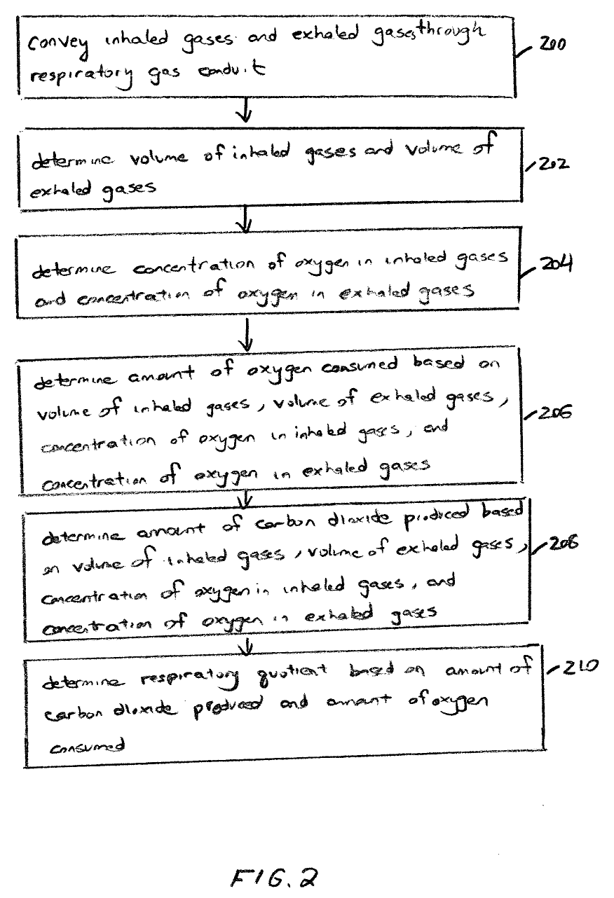

Attention next turns to FIG. 2, which illustrates a flow chart for determining a respiratory quotient RQ of a subject in accordance with an embodiment of the invention. The operations illustrated in FIG. 2 can be performed using a respiratory gas exchange monitor, such as, for example, the respiratory gas exchange monitor 100 previously discussed in connection with FIG. 1.

As illustrated in FIG. 2, a first operation includes conveying inhaled gases and exhaled gases of the subject through a respiratory gas conduit (block 200). In the illustrated embodiment, the inhaled gases and the exhaled gases are conveyed through the respiratory gas conduit by having the subject breathe into and out of the respiratory gas conduit during a test period. As discussed previously, the subject can breathe into and out of the respiratory gas conduit via a respiratory gas connector, which can include a mask or a mouthpiece.

Depending on the specific application, the test period can include a single breath (i.e., one inhalation and one exhalation), various sequential breaths, or various non-sequential breaths spaced over time. Alternatively, or in conjunction, the test period can be specified in units of time, such as, for example, a number of seconds or a number of minutes. Since oxygen and carbon dioxide are transported to and from tissue in the lungs, the composition of respiratory gases in the lungs can vary over time. In particular, during an exhalation, respiratory gases initially expelled from the lungs tend to be more oxygen rich than respiratory gases later expelled from the lungs, which tend to contain relatively more carbon dioxide. Thus, to improve measurement accuracy, the test period desirably includes a sufficient portion of an exhalation, such as, for example, an entire exhalation. For certain applications, determination of the respiratory quotient RQ can be performed by having the subject breathe into and out of the respiratory gas conduit at various times during a day. For example, various respiratory quotients can be determined for test periods associated with mealtimes, administration of medication, exercise schedule, or different times of day, and the various respiratory quotients can be averaged to determine the respiratory quotient RQ.

Referring to FIG. 2, a second operation includes determining a volume of the inhaled gases V_{I} and a volume of the exhaled gases V_{E} (block 202). The volume of the inhaled gases V_{I} can correspond to a volume passing through the respiratory gas conduit during a portion of an inhalation, during a single inhalation, or during multiple inhalations. Similarly, the volume of the exhaled gases V_{E} can correspond to a volume passing through the respiratory gas conduit during a portion of an exhalation, during a single exhalation, or during multiple exhalations. In the illustrated embodiment, the volume of the inhaled gases V_{I} and the volume of the exhaled gases V_{E} are determined based on flow rates of the inhaled gases and the exhaled gases at various times during the test period. As discussed previously, a computation unit can process a set of outputs of a respiratory gas flow meter to determine the flow rates. Based on the flow rates and the known dimensions of the respiratory gas conduit, the computation unit can determine the volume of the inhaled gases V_{I} and the volume of the exhaled gases V_{E}. For certain applications, flow rates can be integrated or summed over the test period to determine the volume of the inhaled gases V_{I} and the volume of the exhaled gases V_{E}.

To allow the volume of the inhaled gases V_{I} and the volume of the exhaled gases V_{E} to be separately determined, the computation unit can process the set of outputs of the respiratory gas flow meter to determine when an inhalation starts or ends or when an exhalation starts or ends. For example, the computation unit can determine whether a flow rate falls below a baseline value to determine a start or an end of an inhalation.

As illustrated in FIG. 2, a third operation includes determining a concentration of oxygen in the inhaled gases F_{I}O₂ and a concentration of oxygen in the exhaled gases F_{E}O₂ (block 204). In the illustrated embodiment, the concentration of oxygen in the exhaled gases F_{E}O₂ is determined based on concentrations of oxygen in the exhaled gases at various times during the test period. As discussed previously, the respiratory gas sensor can be an oxygen sensor, and the computation unit can process a set of outputs of the respiratory gas sensor to determine the concentrations of oxygen in the exhaled gases at various times. For certain applications, the concentrations of oxygen can be averaged over the test period to determine the concentration of oxygen in the exhaled gases F_{E}O₂. The concentration of oxygen in the inhaled gases F_{I}O₂ can be determined in a similar manner as described above for the concentration of oxygen in the exhaled gases F_{E}O₂. Alternatively, or in conjunction, since the inhaled gases are drawn from ambient air, the concentration of oxygen in the inhaled gases F_{I}O₂ can be determined based on a concentration of oxygen in ambient air.

In the illustrated embodiment, the concentration of oxygen in the inhaled gases F_{I}O₂ and the concentration of oxygen in the exhaled gases F_{E}O₂ are represented in volumetric terms. For example, the concentration of oxygen in the inhaled gases F_{I}O₂ can be represented as a volumetric fraction of oxygen in the inhaled gases, and the concentration of oxygen in the exhaled gases F_{E}O₂ can be represented as a volumetric fraction of oxygen in the exhaled gases. It is contemplated that the concentration of oxygen in the inhaled gases F_{I}O₂ and the concentration of oxygen in the exhaled gases F_{E}O₂ can also be represented in molar, mass, or pressure terms.

Referring to FIG. 2, a fourth operation includes determining an amount of oxygen consumed by the subject during the test period based on the volume of the inhaled gases V_{I}, the volume of the exhaled gases V_{E}, the concentration of oxygen in the inhaled gases F_{I}O₂, and the concentration of oxygen in the exhaled gases F_{E}O₂ (block 206). In the illustrated embodiment, the amount of oxygen consumed is represented in volumetric terms, and the computation unit determines the amount of oxygen consumed as a difference between a volume of oxygen in the inhaled gases V_{I}O₂ and a volume of oxygen in the exhaled gases V_{E}O₂. In particular, the amount of oxygen consumed can be represented using the following equation:

Amount of O₂ consumed=V_{I}O₂−V_{E}O₂ (1)

It is contemplated that the amount of oxygen consumed can also be represented in molar, mass, or pressure terms.

In the illustrated embodiment, the volume of oxygen in the inhaled gases V_{I}O₂ is determined by multiplying the volume of the inhaled gases V_{I} by the concentration of oxygen in the inhaled gases F_{I}O₂, and the volume of oxygen in the exhaled gases V_{E}O₂ is determined by multiplying the volume of the exhaled gases V_{E} by the concentration of oxygen in the exhaled gases F_{E}O₂. In particular, the volume of oxygen in the inhaled gases V_{I}O₂ and the volume of oxygen in the exhaled gases V_{E}O₂ can be represented using the following equations:

V_{I}O₂=V_{I}F_{I}O₂V_{E}O₂=V_{E}.F_{E}O₂. (2)

For certain applications, the volume of oxygen in the inhaled gases V_{I}O₂ can be determined based on a concentration of oxygen in ambient air. For example, the concentration of oxygen in the inhaled gases F_{I}O₂ can be assumed to be substantially the same as the concentration of oxygen in ambient air. The volumetric fractions of dry ambient air attributable to carbon dioxide, oxygen, nitrogen, and other inert gases typically do not vary significantly, and typical values of the volumetric fractions are provided in Table I.

TABLE I

| | Volumetric Fraction in Dry |
|---|---|
| Component | Ambient Air |
| CO₂ | 0.00033 |
| O₂ | 0.20946 |
| N₂ and other inert gases | 0.79021 |

In some instances, the inhaled gases do not correspond to dry ambient air. Rather, the inhaled gases can include a volumetric fraction attributable to water vapor that can vary from location to location as well as from time to time. To determine the volume of oxygen in the inhaled gases V_{I}O₂, a volume of water vapor in the inhaled gases V_{I}H₂O can be determined and subtracted from the volume of the inhaled gases V_{I} to obtain a dry volume of the inhaled gases V_{I,dry}. For certain applications, the volume of water vapor in the inhaled gases V_{I}H₂O can be determined based on a pressure P_{I}, a relative humidity RH_{I}, and a temperature T_{I} of the inhaled gases. In particular, the volume of water vapor in the inhaled gases V_{I}H₂O can be represented using the following equation:

V_{I}H₂O=f(P_{I}, RH_{I}, T_{I}), (3)

where f corresponds to a function that can be represented using, for example, an empirical curve fit or a look-up table. As one of ordinary skill in the art will understand, the function f can be determined based on a vapor pressure of water. As discussed previously, an environmental sensor can generate a set of outputs associated with the pressure P_{I}, the relative humidity RH_{I}, and the temperature T_{I}, and the computation unit can process the set of outputs of the environmental sensor to determine the volume of water vapor in the inhaled gases V_{I}H₂O. It is contemplated that the computation unit can perform corrections to the set of outputs to account for heating effects, such as, for example, those resulting from contact with the subject or operation of hardware circuitry. It is also contemplated that the computation unit can determine one or more of the pressure P_{I}, the relative humidity RH_{I}, and the temperature T_{I} based on corresponding values in ambient air. For example, the pressure P_{I}, the relative humidity RH_{I}, and the temperature T_{I} can be assumed to be substantially the same as the corresponding values in ambient air. It is further contemplated that water vapor can be removed from the inhaled gases, such that the inhaled gases can correspond to dry ambient air. For example, water vapor can be removed from the inhaled gases using a desiccant.

Once the dry volume of the inhaled gases V_{I,dry} is determined, the volume of oxygen in the inhaled gases V_{I}O₂ can be determined by multiplying the dry volume of the inhaled gases V_{I,dry} with a concentration of oxygen in dry ambient air F_{I,dry}O₂. In particular, the volume of oxygen in the inhaled gases V_{I}O₂ can be represented using the following equation:

V_{I}O₂=V_{I,dry}.F_{I,dry}O₂, (4)

where the concentration of oxygen in dry ambient air F_{I,dry}O₂ corresponds to a volumetric fraction of oxygen in dry ambient air.

As illustrated in FIG. 2, a fifth operation includes determining an amount of carbon dioxide produced by the subject during the test period based on the volume of the inhaled gases V_{I}, the volume of the exhaled gases V_{E}, the concentration of oxygen in the inhaled gases F_{I}O₂, and the concentration of oxygen in the exhaled gases F_{E}O₂ (block 208). In the illustrated embodiment, the amount of carbon dioxide produced is represented in volumetric terms, and the computation unit determines the amount of carbon dioxide produced as a difference between a volume of carbon dioxide in the exhaled gases V_{E}CO₂ and a volume of carbon dioxide in the inhaled gases V_{I}CO₂. In particular, the amount of carbon dioxide produced can be represented using the following equation:

Amount of CO₂ produced=V_{E}CO₂−V_{I}CO₂. (5)

It is contemplated that the amount of carbon dioxide produced can also be represented in molar, mass, or pressure terms.

Typically, nitrogen and other inert gases present in the inhaled gases and the exhaled gases are neither consumed nor produced by the subject. Thus, volumes attributable to nitrogen and the other inert gases can be ignored when determining the amount of carbon dioxide produced. However, the inhaled gases and the exhaled gases can include different volumetric fractions of water vapor. To determine the volume of carbon dioxide in the exhaled gases V_{E}CO₂, a volume of water vapor in the exhaled gases V_{E}H₂O can be determined and subtracted from the volume of the exhaled gases V_{E} to obtain a dry volume of the exhaled gases V_{E,dry}. For certain applications, the volume of water vapor in the exhaled gases V_{E}H₂O can be determined based on a pressure P_{E}, a relative humidity RH_{E}, and a temperature T_{E} of the exhaled gases. In particular, the volume of water vapor in the exhaled gases V_{E}H₂O can be represented using the following equation:

V_{E}H₂O=f(_{P}E, RH_{E}, T_{E}), (6)

where f corresponds to the function previously discussed in connection with equation (3). As discussed previously, the environmental sensor can generate a set of outputs associated with the pressure P_{E}, the relative humidity RH_{E}, and the temperature T_{E}, and the computation unit can process the set of outputs of the environmental sensor to determine the volume of water vapor in the exhaled gases V_{E}H₂O. It is contemplated that the computation unit can perform corrections to the set of outputs to account for heating effects. It is also contemplated that the computation unit can determine one or more of the pressure P_{E}, the relative humidity RH_{E}, and the temperature T_{E} based on physiological parameters of the subject. For example, one or more of the pressure P_{E}, the relative humidity RH_{E}, and the temperature T_{E} can be assumed to be substantially the same as the corresponding values in a typical exhalation. It is further contemplated that water vapor can be added to or removed from the inhaled gases and exhaled gases, such that the inhaled gases and the exhaled gases can include substantially the same volumetric fraction of water vapor. For example, water vapor can be added using a water-containing structure such as a soaked sponge or removed using a desiccant.

Once the dry volume of the exhaled gases V_{E,dry} is determined, the volume of carbon dioxide in the exhaled gases V_{E}CO₂ can be determined by subtracting the volume of oxygen in the exhaled gases V_{E}O₂ from the dry volume of the exhaled gases V_{E,dry.} In particular, the volume of carbon dioxide in the exhaled gases V_{E}CO₂ can be represented using the following equation:

V_{E}CO₂=V_{E,dry}−V_{E}O₂, (7)

where the volumes attributable to nitrogen and the other inert gases have been omitted.

Similarly, the volume of carbon dioxide in the inhaled gases V_{I}CO₂ can be determined by subtracting the volume of oxygen in the inhaled gases V_{I}O₂ from the dry volume of the inhaled gases V_{I,dry}. In particular, the volume of carbon dioxide in the inhaled gases V_{I}CO₂ can be represented using the following equation:

V_{I}CO₂=V_{I,dry}−V_{I}O₂, (8)

where the volumes attributable to nitrogen and the other inert gases have been omitted.

As illustrated in FIG. 2, a sixth operation includes determining the respiratory quotient RQ of the subject based on the amount of carbon dioxide produced by the subject during the test period and the amount of oxygen consumed by the subject during the test period (block 210). In the illustrated embodiment, the computation unit determines the respiratory quotient RQ as a ratio of the amount of carbon dioxide produced and the amount of oxygen consumed. In particular, the respiratory quotient can be represented using the following equation:

RQ=(Amount of CO₂ produced)/(Amount of O₂ consumed) (9)

In some instances, the inhaled gases and the exhaled gases can have different temperatures. For example, the temperature T_{I} can be substantially the same as a temperature of ambient air, while the temperature T_{E} can be substantially the same as a body temperature. Since temperature can affect volumes of respiratory gases, the computation unit desirably normalizes the volumes of the respiratory gases with respect to a common temperature to facilitate direct comparison. For example, in connection with determining the amount of oxygen consumed in equation (1), the computation unit desirably performs a normalization of the volume of oxygen in the inhaled gases V_{I}O₂ and the volume of oxygen in the exhaled gases V_{E}O₂ with respect to a common temperature, such as, for example, 25° C. or 37° C. Similarly, in connection with determining the amount of carbon dioxide produced in equation (5), the computation unit desirably performs a normalization of the volume of carbon dioxide in the exhaled gases V_{E}CO₂ and the volume of carbon dioxide in the inhaled gases V_{I}CO₂ with respect to the same common temperature. Since pressure can also affect volumes of respiratory gases, the computation unit can also normalize the volumes of the respiratory gases with respect to a common pressure to facilitate direct comparison. For example, the computation unit can normalize volumes of the respiratory gases with respect to a common pressure, such as, for example, 1 atm. As discussed previously, the environmental sensor can generate a set of outputs associated with the pressure P_{I}, the temperature T_{I}, the pressure P_{E}, and the temperature T_{E}, and the computation unit can process the set of outputs of the environmental sensor to normalize volumes of respiratory gases. It is contemplated that the computation unit can determine one or more of the pressure P_{I}, the temperature T_{I}, the pressure P_{E}, and the temperature T_{E} based on assumptions associated with ambient air or the subject. It is further contemplated that volumes of respiratory gases can be normalized by changing a temperature or a pressure of the respiratory gases to a common temperature or a common pressure. For example, a temperature of the respiratory gases can be changed using a heating or cooling element, such as, for example, a Peltier element.

While FIG. 2 is discussed with reference to determining concentrations of oxygen in respiratory gases, it is contemplated that a similar set of operations can be performed based on determining concentrations of carbon dioxide in the respiratory gases. For example, the respiratory gas sensor can be a carbon dioxide sensor, and the computation unit can process a set of outputs of the respiratory gas sensor to determine a concentration of carbon dioxide in the inhaled gases F_{I}CO₂ and a concentration of carbon dioxide in the exhaled gases F_{E}CO₂. In particular, the concentration of carbon dioxide in the inhaled gases F_{I}CO₂ and the concentration of carbon dioxide in the exhaled gases F_{E}CO₂ can be determined in a similar manner as described above for oxygen. Next, the volume of carbon dioxide in the inhaled gases V_{I}CO₂ can be determined by multiplying the volume of the inhaled gases V_{I} with the concentration of carbon dioxide in the inhaled gases F_{I}CO₂, and the volume of carbon dioxide in the exhaled gases V_{E}CO₂ can be determined by multiplying the volume of the exhaled gases V_{E} with the concentration of carbon dioxide in the exhaled gases F_{E}CO₂. Based on the volume of carbon dioxide in the inhaled gases V_{I}CO₂ and the volume of carbon dioxide in the exhaled gases V_{E}CO₂, the amount of carbon dioxide produced, the amount of oxygen consumed, and the respiratory quotient RQ can be determined in a similar manner as described above in connection with FIG. 2.

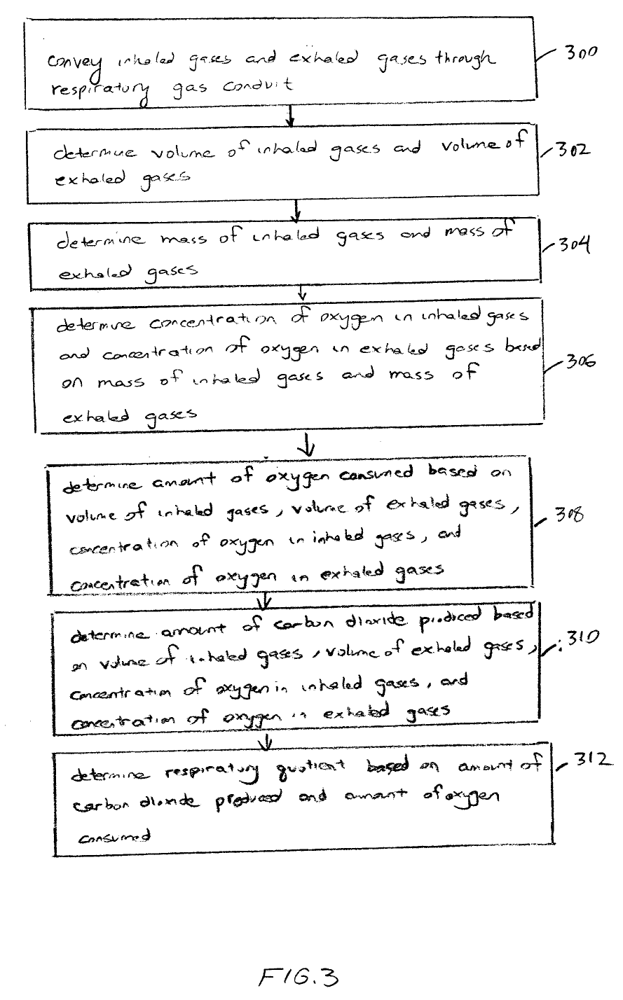

FIG. 3 illustrates a flow chart for determining a respiratory quotient RQ of a subject in accordance with another embodiment of the invention. The operations illustrated in FIG. 3 can be performed using a respiratory gas exchange monitor, such as, for example, the respiratory gas exchange monitor 100 previously discussed in connection with FIG. 1. Advantageously, the operations illustrated in FIG. 3 can be performed using a respiratory gas exchange monitor that need not include a respiratory gas sensor.

As illustrated in FIG. 3, a first operation includes conveying inhaled gases and exhaled gases of the subject through a respiratory gas conduit (block 300), and a second operation includes determining a volume of the inhaled gases V_{I} and a volume of the exhaled gases V_{E} (block 302). In the illustrated embodiment, the first and second operations can be performed in a similar manner as described above in connection with FIG. 2. For example, the inhaled gases and the exhaled gases can be conveyed through the respiratory gas conduit by having the subject breathe into and out of the respiratory gas conduit during a test period.

Referring to FIG. 3, a third operation includes determining a mass of the inhaled gases M_{I} and a mass of the exhaled gases M_{E} (block 304). Depending on the specific application, the mass of the inhaled gases M_{I} and the mass of the exhaled gases M_{E} can be represented in absolute or molar terms. In the illustrated embodiment, the mass of the exhaled gases M_{E} is represented in absolute terms and is determined based on densities of the exhaled gases at various times during the test period. As discussed previously, the respiratory gas flow meter can include a pair of spaced apart piezoelectric crystal transducers, and the computation unit can process a set of outputs of the respiratory gas flow meter to determine the densities of the exhaled gases at various times. Based on the densities of the exhaled gases, the computation unit can determine the mass of the exhaled gases M_{E}. For certain applications, the densities of the exhaled gases can be averaged over the test period and multiplied with the volume of the exhaled gases V_{E} to determine the mass of the exhaled gases M_{E}. For other applications, the densities of the exhaled gases can be multiplied with associated flow rates and then integrated or summed over the test period to determine the mass of the exhaled gases M_{E}.

In the illustrated embodiment, the mass of the inhaled gases M_{I} is also represented in absolute terms and can be determined in a similar manner as described above for the mass of the exhaled gases M_{E}. Alternatively, the mass of the inhaled gases M_{I} need not be determined, since the respiratory quotient RQ can be determined based on other respiratory parameters as further described below.

It is contemplated that the mass of the inhaled gases M_{I} and the mass of the exhaled gases M_{E} can also be represented in molar terms (i.e., as molar masses) and can be determined based on “upstream” and “downstream” transit times. As discussed previously, ultrasonic pulses can be transmitted with and against a direction along which respiratory gases pass through the respiratory gas conduit, resulting in a set of outputs associated with “upstream” and “downstream” transit times. The mass of the inhaled gases M_{I} and the mass of the exhaled gases M_{E} can be determined based on a speed of sound, which, in turn, can be determined based on the “upstream” and “downstream” transit times. Additional discussion regarding determining a molar mass based on “upstream” and “downstream” transit times can be found, for example, in U.S. Pat. No. 5,645,071, the disclosure of which is incorporated herein by reference in its entirety.

As illustrated in FIG. 3, a fourth operation includes determining a concentration of oxygen in the inhaled gases F_{I}O₂ and a concentration of oxygen in the exhaled gases F_{E}O₂ based on the mass of the inhaled gases M_{I} and the mass of the exhaled gases M_{E} (block 306). In the illustrated embodiment, the concentration of oxygen in the exhaled gases F_{E}O₂ is determined based on the mass of the exhaled gases M_{E}. To determine the concentration of oxygen in the exhaled gases F_{E}O₂, a mass of nitrogen in the exhaled gases M_{E}N₂, a mass of the other inert gases in the exhaled gases M_{E}other, and a mass of water vapor in the exhaled gases M_{E}H₂O can be determined and subtracted from the mass of the exhaled gases M_{E} to obtain a mass of carbon dioxide and oxygen in the exhaled gases M_{E}CO₂&O₂. In the illustrated embodiment, the mass of carbon dioxide and oxygen in the exhaled gases M_{E}CO₂&O₂ can be represented using the following equation:

M_{E}CO₂&O₂=g(F_{E}O₂, F_{E}CO₂) (10)

where g corresponds to a function that can be represented using, for example, an empirical curve fit or a look-up table. Since carbon dioxide has a greater mass than oxygen, substitution of carbon dioxide for oxygen in the exhaled gases can lead to an increase in the mass of carbon dioxide and oxygen in the exhaled gases M_{E}CO₂&O₂. On the other hand, substitution of oxygen for carbon dioxide in the exhaled gases can lead to a decrease in the mass of carbon dioxide and oxygen in the exhaled gases M_{E}CO₂&O₂. In some instances, the function g can be linearly related to a relative proportion of carbon dioxide and oxygen in the exhaled gases, and the concentration of oxygen in the exhaled gases F_{E}O₂ as well as the concentration of carbon dioxide in the exhaled gases F_{E}CO₂ can be determined based on equation (10).

As discussed previously, nitrogen and the other inert gases present in the inhaled gases and the exhaled gases are typically neither consumed nor produced by the subject, while water vapor can be present in different amounts in the inhaled gases and the exhaled gases. Thus, the mass of nitrogen in the exhaled gases M_{E}N₂ and the mass of the other inert gases in the exhaled gases M_{E}other can be determined to be substantially the same as their counterparts in the inhaled gases, which can be determined based on a standard gas equation and concentrations of nitrogen and the inert gases in ambient air. For certain applications, the mass of water vapor in the exhaled gases M_{E}H₂O can be determined based on the mass of the exhaled gases M_{E}, the pressure P_{E}, the relative humidity RH_{E}, and the temperature T_{E}. In particular, the mass of water vapor in the exhaled gases M_{E}H₂O can be represented using the following equation:

M_{E}H₂O=M_{E}.h(P_{E}, RH_{E}, T_{E}), (11)

where h corresponds to a function that can be represented using, for example, an empirical curve fit or a look-up table. As one of ordinary skill in the art will understand, the function h can be determined based on a humidity ratio of water.

The concentration of oxygen in the inhaled gases F_{I}O₂ can be determined in a similar manner as described above for the concentration of oxygen in the exhaled gases F_{E}O₂. Thus, for example, to determine the concentration of oxygen in the inhaled gases F_{I}O₂, a mass of nitrogen in the inhaled gases M_{I}N₂, a mass of the other inert gases in the inhaled gases M_{I}other, and a mass of water vapor in the inhaled gases M_{I}H₂O can be determined and subtracted from the mass of the inhaled gases M_{I} to obtain a mass of carbon dioxide and oxygen in the inhaled gases M_{I}CO₂&O₂. In the illustrated embodiment, the mass of carbon dioxide and oxygen in the inhaled gases M_{I}CO₂&O₂ can be represented using the following equation:

M_{I}CO₂&O₂=g(F_{I}O₂, F_{I}CO₂), (12)

where g corresponds to the function previously discussed in connection with equation (10). As discussed previously, the function g can be linearly related to a relative proportion of carbon dioxide and oxygen in the inhaled gases, and the concentration of oxygen in the inhaled gases F_{I}O₂ as well as the concentration of carbon dioxide in the inhaled gases F_{I}CO₂ can be determined based on equation (12). Alternatively, or in conjunction, since the inhaled gases are drawn from ambient air, the concentration of oxygen in the inhaled gases F_{I}O₂ can be determined based on a concentration of oxygen in ambient air.

Referring to FIG. 3, a fifth operation includes determining an amount of oxygen consumed by the subject during the test period based on the volume of the inhaled gases V_{I}, the volume of the exhaled gases V_{E}, the concentration of oxygen in the inhaled gases F_{I}O₂, and the concentration of oxygen in the exhaled gases F_{E}O₂ (block 308). Also, a sixth operation includes determining an amount of carbon dioxide produced by the subject during the test period based on the volume of the inhaled gases V_{I}, the volume of the exhaled gases V_{E}, the concentration of oxygen in the inhaled gases F_{I}O₂, and the concentration of oxygen in the exhaled gases F_{E}O₂ (block 310). And, a seventh operation includes determining the respiratory quotient RQ of the subject based on the amount of carbon dioxide produced by the subject during the test period and the amount of oxygen consumed by the subject during the test period (block 312). In the illustrated embodiment, the fifth, the sixth, and the seventh operations can be performed in a similar manner as described above in connection with FIG. 2. In connection with the sixth operation, it is contemplated that the amount of carbon dioxide produced can also be determined based on the volume of the inhaled gases V_{I}, the volume of the exhaled gases V_{E}, the concentration of carbon dioxide in the inhaled gases F_{I}CO₂, and the concentration of carbon dioxide in the exhaled gases F_{E}CO₂.

It should be recognized that the specific embodiments of the invention described above are provided by way of example, and various other embodiments are encompassed by the invention.

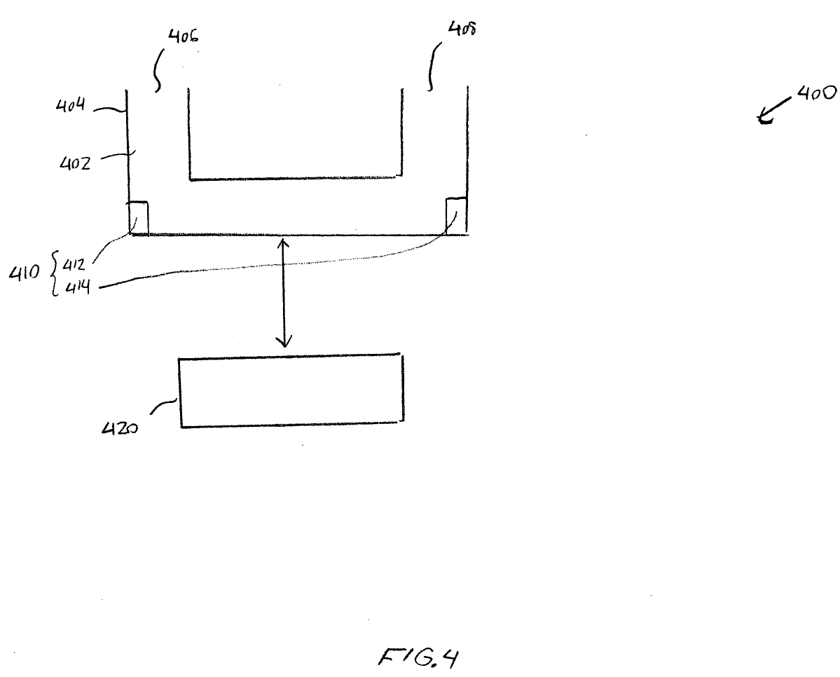

For example, FIG. 4 illustrates a respiratory gas exchange monitor 400 implemented in accordance with another embodiment of the invention. The respiratory gas exchange monitor 400 can be operated to analyze respiratory gases of a subject to determine a respiratory quotient of the subject. Advantageously, the respiratory gas exchange monitor 400 can be used to determine the respiratory quotient without requiring the use of a respiratory gas sensor. In particular, the respiratory gas exchange monitor 400 can be used to perform the operations illustrated in FIG. 3. As illustrated in FIG. 4, the respiratory gas exchange monitor 400 includes a respiratory gas conduit 402, which includes a flow tube 404. In the illustrated embodiment, the flow tube 404 is U-shaped and includes a pair of openings 406 and 408. The opening 406 is configured to interface with the subject so as to provide inhaled gases to the subject and to receive exhaled gases from the subject, while the opening 408 is configured to interface with ambient air. The respiratory gas exchange monitor 400 also includes a respiratory gas flow meter or sensor 410, which is coupled to the respiratory gas conduit 402. In the illustrated embodiment, the respiratory gas flow meter 410 is desirably an ultrasonic flow meter and includes a pair of spaced apart ultrasonic transducers 412 and 414. As illustrated in FIG. 4, the respiratory gas exchange monitor 400 also includes a computation unit 420, which is coupled to the respiratory gas flow meter 410.

An embodiment of the invention relates to determining a respiratory quotient of a subject who is incapable or prohibited from self-feeding, such as, for example, an unconscious person, an infant, or a recovering surgical patient. Typically, the nutritional requirements of such subject are provided by enteral administration, such as via epigastric tubing, or by parenteral administration, such as via intravenous injection. To monitor the nutritional status of the subject, respiratory quotients can be determined at regular or irregular intervals. In certain situations, the subject may be incapable of operating a respiratory gas exchange monitor, and a healthcare professional can operate the respiratory gas exchange monitor to determine respiratory quotients. The respiratory gas exchange monitor can also be attached to a supporting structure or retained next to the subject's mouth by an attachment device, such as, for example, a strap wrapped around the head of the subject. A reference respiratory quotient can be determined based on nutrient intake of the subject and is typically in the range of about 0.8 to about 0.9. In particular, the reference respiratory quotient can be determined based on the amounts and types of food items ingested by the subject. In some instances, the reference respiratory quotient can be determined based on a typical diet of mixed food items. Respiratory quotients for various food items can be determined based on tabulated values as, for example, described in Lovesey and Elia, “Estimation of Energy Expenditure, Net Carbohydrate Utilization, and Net Fat Oxidation and Synthesis by Indirect Calorimetry: Evaluation of Errors with Special Reference to the Detailed Composition of Fuels,” Am. J. Clin. Nutr., 1988, 47:608-28. Higher or lower values of a respiratory quotient with respect to the reference respiratory quotient can indicate an undesirable imbalance in administered nutrient intake or an inability to absorb or metabolize a particular metabolic substrate. In some instances, the reference respiratory quotient can be specified by a user, such as, for example, using a data entry device. Alternatively, or in conjunction, a reference respiratory quotient range can be determined based on nutrient intake of the subject or can be specified by a user. A respiratory quotient can be determined and compared with the reference respiratory quotient, such that a measure of deviation with respect to the reference respiratory quotient can be determined. The measure of deviation can be stored in a memory for later processing. Additionally, the measure of deviation can be used to trigger a warning signal, such as, for example, a tone, a light, or a displayed verbal warning. A warning signal can also include directives or feedback. For example, if there is significant deviation from the reference respiratory quotient, the subject may be advised not to eat certain food items. In some instances, a warning signal can be transmitted to another location, such as, for example, a nursing station, a physician's office, a paging device, or an electronic medical file. Once alerted, a healthcare professional can investigate the problem and appropriately adjust the nutrient intake of the subject. For certain applications, the subject's respiratory quotient can be compared with an analysis of actual nutrient intake to establish a provisional determination of whether the measure of deviation is nutritionally based or has another cause, such as, for example, a metabolic imbalance, a pulmonary dysfunction, or a recent physical exertion.

An embodiment of the invention relates to determining a respiratory quotient of a subject whose breathing is assisted, such as, for example, using a mechanical ventilator. A ventilated subject may be incapable of delivering respiratory gases into a respiratory gas conduit. In such situations, a respiratory gas exchange monitor can be attached to the mechanical ventilator, such that afferent as well as efferent respiratory gases can pass through the respiratory gas conduit for determining the respiratory quotient.

An embodiment of the invention relates to determining a respiratory quotient of a subject suspected of having a metabolic disorder. As discussed previously, a respiratory quotient can be indicative of oxidative metabolism in cells. Higher or lower values of a respiratory quotient with respect to a reference respiratory quotient can indicate a metabolic disorder. For example, a higher value of a respiratory quotient can occur in subjects suffering from, for example, hypothyroidism. As another example, a higher value of a respiratory quotient can be the result of metabolic acidosis associated with uncontrolled insulin-dependent diabetes or kidney failure. Thus, determining a respiratory quotient can be used to diagnose metabolic disorders.

An embodiment of the invention relates to determining a respiratory quotient of a subject to monitor compliance with a dietary regimen. A respiratory quotient can be a valuable indication of nutrient intake of a self-feeding subject and can be used to monitor nutritional status of the subject. For example, a subject that is overweight can be advised to maintain a balanced dietary regimen without an overabundance of carbohydrates. The subject can be advised that the balanced dietary regimen should produce a target respiratory quotient in the range of about 0.8 to about 0.85. Compliance with the dietary regimen can be increased if the subject can obtain feedback with little inconvenience. Thus, in accordance with an embodiment of the invention, the subject desiring to achieve the target respiratory quotient can determine a respiratory quotient as described herein and can compare the respiratory quotient with the target respiratory quotient to determine a measure of deviation, and the measure of deviation can be used to monitor compliance with the dietary regimen.

An embodiment of the invention relates to storing values of respiratory quotients in a database. Such storing can be performed daily or according to a schedule specified by a healthcare professional. For example, when monitoring compliance with a dietary regimen, a target respiratory quotient can be set. Following analysis of a breath or series of breaths, a value of a respiratory quotient can be stored in the database along with other indicia, such as, for example, name of a subject, date, and time. In addition, a value of a measure of deviation with respect to the target respiratory quotient can be determined and stored in the database. Stored information can be provided to a user or a healthcare professional in the form of, for example, a table or a graph. In addition, additional information relevant to respiratory quotients can be entered to monitor behavior of a subject, such as, for example, compliance with a dietary regimen. For example, the subject may wish to control carbohydrate intake to control weight or to control a metabolic disorder such as diabetes. Information relating to the types and amounts of food items eaten or desired to be eaten can be stored in the database. In some instances, an expected respiratory quotient can be determined based on the stored information. Additional information relevant to respiratory quotients can be stored in the database and can include physiological parameters, such as, for example, a subject's daily exercise activities, therapeutic drug intake, blood glucose level, excreted nitrogen level, excreted ketone level, lactate level, heart rate, ventilation rate, body temperature, metabolic rate, and hydration level.

Another embodiment of the invention relates to determining a respiratory quotient of a subject during exercise. Such determination can serve to optimize athletic performance and avoid an energy depletion episode. Endurance athletes can sometimes undergo an energy depletion episode during long-term exercise, which can be characterized by severe fatigue and is common referred to as “hitting the wall.” An energy depletion episode can arise as a result of depletion of stored carbohydrates such as glycogen. Carbohydrates can constitute the major metabolic substrate during exercise. Metabolism of carbohydrates is typically aerobic and can involve the consumption of oxygen and the production of carbon dioxide, water, and ATP. However, during endurance exercise, cells can also metabolize carbohydrates in the absence of oxygen, resulting in the production of adenosine triphosphate and lactate. The transition between predominantly aerobic metabolism and predominantly anaerobic metabolism is commonly referred to as the “anaerobic threshold.” The point at which lactate starts to increase in the blood stream is called the “lactate threshold” and can be used as an approximation of the “anaerobic threshold.” In an intensely exercising individual, lactate can build up in the blood stream as cells are unable to process lactate quickly enough. Lactate in the blood stream can lower the blood pH, so that bicarbonate can be called upon as a buffer. During such buffering process, carbon dioxide can be produced. This carbon dioxide can be distinguished from that produced during cellular respiration. Thus, in an exercising individual, a point can be reached when a portion of carbon dioxide produced reflects anaerobic metabolism. Typically, when determining a respiratory quotient during exercise, a rise in the respiratory quotient can occur near the “anaerobic threshold,” making it possible to determine the onset of anaerobic metabolism. Advantageously, determining a respiratory quotient during exercise can be used to inform an athlete regarding his or her current metabolic state, so that the athlete can modify or discontinue exercise. For example, an endurance athlete training to run long distances may wish to run a particular distance without having to stop due to exhaustion. Respiratory quotients of the athlete can be determined at various times during exercise using a respiratory gas exchange monitor. The respiratory quotients can be stored in a memory if desired. Typically, a respiratory quotient of an individual exercising at low intensity for shorter durations will have a lower value than that typically observed for an individual on an average diet, since lipids can be a major metabolic substrate when exercising at low intensity. However, with prolonged exercise, the respiratory quotient can increase as carbohydrates become a major metabolic substrate, and this increase can be proportional to an increase in the amount of oxygen consumed. With further exercise, the respiratory quotient can increase independently of a proportional increase in the amount of oxygen consumed, and carbon dioxide can be produced during lactate buffering. A warning signal can be triggered when the respiratory quotient increases beyond a certain level. Another warning signal can be triggered by a further increase in the respiratory quotient, so that the athlete can modify or discontinue exercise without “hitting the wall.”

An embodiment of the invention relates to using a respiratory quotient to monitor oxidation of a particular metabolic substrate during exercise. For example, a subject may desire to oxidize or “burn” lipids during exercise. In this example, the subject may set a target respiratory quotient to be in the range of about 0.7 to about 0.75 during exercise. In some instances, respiratory quotients are determined during exercise, and a warning signal is triggered when the target respiratory quotient is reached. Significant deviations from the target respiratory quotient can trigger another warning signal, so that the subject can be alerted to modify or discontinue exercise.

A practitioner of ordinary skill in the art should require no additional explanation in developing the invention described and claimed herein but may nevertheless find some helpful guidance by examining U.S. Pat. Nos. 5,836,300; 6,135,107; 6,277,645; 6,309,360; 6,402,698; 6,406,435; and 6,506,608; the disclosures of which are incorporated herein by reference in their entirety.

Each of the patent applications, patents, publications, and other published documents mentioned or referred to in this specification is herein incorporated by reference in its entirety, to the same extent as if each individual patent application, patent, publication, and other published document was specifically and individually indicated to be incorporated by reference.

While the invention has been described with reference to the specific embodiments thereof, it should be understood by those skilled in the art that various changes may be made and equivalents may be substituted without departing from the true spirit and scope of the invention as defined by the appended claims. In addition, many modifications may be made to adapt a particular situation, material, composition of matter, method, or operation to the objective, spirit and scope of the invention. All such modifications are intended to be within the scope of the claims appended hereto. In particular, while the methods disclosed herein have been described with reference to particular operations performed in a particular order, it will be understood that these operations may be combined, sub-divided, or re-ordered to form an equivalent method without departing from the teachings of the invention. Accordingly, unless specifically indicated herein, the order and grouping of the operations is not a limitation of the invention.

What is claimed is:

1. A respiratory gas exchange monitor, comprising:
a respiratory gas conduit configured to convey inhaled gases and exhaled gases of a subject;
a respiratory gas flow meter coupled to said respiratory gas conduit, said respiratory gas flow meter being configured to generate an output associated with a volume of said inhaled gases and a volume of said exhaled gases;
a respiratory gas sensor coupled to said respiratory gas conduit, said respiratory gas sensor being configured to generate an output associated with a concentration of oxygen in said exhaled gases; and
a computation unit coupled to said respiratory gas flow meter and said respiratory gas sensor, said computation unit being configured to process said output of said respiratory gas flow meter and said output of said respiratory gas sensor to determine an amount of carbon dioxide produced by said subject and an amount of oxygen consumed by said subject, said computation unit being configured to determine a respiratory quotient of said subject based on said amount of carbon dioxide produced and said amount of oxygen consumed.

2. The respiratory gas exchange monitor of claim 1, wherein said respiratory gas flow meter is an ultrasonic flow meter.

3. The respiratory gas exchange monitor of claim 1, wherein said respiratory gas sensor is an oxygen sensor.

4. The respiratory gas exchange monitor of claim 1, wherein said output of said respiratory gas sensor is further associated with a concentration of oxygen in said inhaled gases.

5. The respiratory gas exchange monitor of claim 1, wherein said computation unit is configured to process said output of said respiratory gas flow meter to determine said volume of said inhaled gases and said volume of said exhaled gases, and said computation unit is configured to process said output of said respiratory gas sensor to determine said concentration of oxygen in said exhaled gases.

6. The respiratory gas exchange monitor of claim 5, wherein said computation unit is configured to determine said amount of carbon dioxide produced and said amount of oxygen consumed based on said volume of said inhaled gases, said volume of said exhaled gases, said concentration of oxygen in said exhaled gases, and a concentration of oxygen in said inhaled gases.

7. The respiratory gas exchange monitor of claim 6, wherein said computation unit is configured to determine said concentration of oxygen in said inhaled gases based on a concentration of oxygen in ambient air.

8. The respiratory gas exchange monitor of claim 6, wherein said computation unit is configured to determine said respiratory quotient based on a ratio of said amount of carbon dioxide produced and said amount of oxygen consumed.

9. The respiratory gas exchange monitor of claim 1, wherein said computation unit is configured to compare said respiratory quotient with a reference respiratory quotient to determine a measure of deviation of said respiratory quotient with respect to said reference respiratory quotient.

10. The respiratory gas exchange monitor of claim 9, wherein said computation unit is configured to determine said reference respiratory quotient based on a nutrient intake of said subject.

11. The respiratory gas exchange monitor of claim 1, further comprising a display unit coupled to said computation unit, said display unit being configured to provide indicia of said respiratory quotient.

12. A respiratory gas exchange monitor, comprising:
a respiratory gas flow meter configured to generate an output associated with inhaled gases and exhaled gases of a subject;
a respiratory gas sensor configured to generate an output associated with said exhaled gases; and
a computation unit coupled to said respiratory gas flow meter and said respiratory gas sensor, said computation unit being configured to process said output of said respiratory gas flow meter to determine a volume of said inhaled gases and a volume of said exhaled gases, said computation unit being configured to process said output of said respiratory gas sensor to determine a concentration of oxygen in said exhaled gases, said computation unit being configured to determine an amount of carbon dioxide produced by said subject and an amount of oxygen consumed by said subject based on said volume of said inhaled gases, said volume of said exhaled gases, and said concentration of oxygen in said exhaled gases, said computation unit being configured to determine a respiratory quotient of said subject based on a ratio of said amount of carbon dioxide produced and said amount of oxygen consumed.

13. The respiratory gas exchange monitor of claim 12, further comprising a respiratory gas conduit configured to convey said inhaled gases and said exhaled gases as said subject breathes, said respiratory gas flow meter and said respiratory gas sensor being coupled to said respiratory gas conduit.

14. The respiratory gas exchange monitor of claim 13, wherein said respiratory gas conduit includes a flow tube.

15. The respiratory gas exchange monitor of claim 12, wherein said respiratory gas flow meter includes a plurality of ultrasonic transducers.

16. The respiratory gas exchange monitor of claim 12, wherein said respiratory gas sensor is a fluorescence quench oxygen sensor.

17. The respiratory gas exchange monitor of claim 12, further comprising a display unit coupled to said computation unit, said display unit being configured to provide indicia of said respiratory quotient.

18. A respiratory gas exchange monitor, comprising:
a respiratory gas flow meter configured to generate an output associated with inhaled gases and exhaled gases of a subject; and
a computation unit coupled to said respiratory gas flow meter, said computation unit being configured to process said output of said respiratory gas flow meter to determine a volume of said inhaled gases, a volume of said exhaled gases, and a mass of said exhaled gases, said computation unit being configured to determine an amount of carbon dioxide produced by said subject and an amount of oxygen consumed by said subject based on said volume of said inhaled gases, said volume of said exhaled gases, and said mass of said exhaled gases, said computation unit being configured to determine a respiratory quotient of said subject based on a ratio of said amount of carbon dioxide produced and said amount of oxygen consumed.

19. The respiratory gas exchange monitor of claim 18, further comprising a respiratory gas conduit configured to convey said inhaled gases and said exhaled gases as said subject breathes, said respiratory gas flow meter being coupled to said respiratory gas conduit.

20. The respiratory gas exchange monitor of claim 18, wherein said respiratory gas conduit includes a flow tube.

21. The respiratory gas exchange monitor of claim 18, wherein said respiratory gas flow meter includes a plurality of ultrasonic transducers.

22. The respiratory gas exchange monitor of claim 18, wherein said computation unit is configured to determine a mass of carbon dioxide and oxygen in said exhaled gases based on said mass of said exhaled gases and a mass of nitrogen in said exhaled gases.

23. The respiratory gas exchange monitor of claim 22, wherein said computation unit is configured to determine said mass of nitrogen in said exhaled gas based on a concentration of nitrogen in ambient air.

24. The respiratory gas exchange monitor of claim 22, wherein said computation unit is configured to determine a concentration of oxygen in said exhaled gases based on said mass of carbon dioxide and oxygen in said exhaled gases, and said computation unit is configured to determine said amount of carbon dioxide produced and said amount of oxygen consumed based on said volume of said inhaled gases, said volume of said exhaled gases, and said concentration of oxygen in said exhaled gases.

25. The respiratory gas exchange monitor of claim 18, further comprising a display unit coupled to said computation unit, said display unit being configured to provide indicia of said respiratory quotient.

26. A respiratory gas exchange monitor, comprising:
a conduit configured to convey inhaled gases and exhaled gases of a subject;
a first sensor coupled to said conduit, said first sensor being configured to generate a first signal associated with a volume of said inhaled gases and a volume of said exhaled gases;
a second sensor coupled to said conduit, said second sensor being configured to generate a second signal associated with a concentration of oxygen in said exhaled gases; and
a computation unit coupled to said first sensor and said second sensor, said computation unit being configured to process said first signal and said second signal to determine an amount of carbon dioxide produced by said subject and an amount of oxygen consumed by said subject, said computation unit being configured to determine a respiratory quotient of said subject based on said amount of carbon dioxide produced and said amount of oxygen consumed.

27. The respiratory gas exchange monitor of claim 26, wherein said conduit includes a flow tube.

28. The respiratory gas exchange monitor of claim 26, wherein said first sensor is an ultrasonic flow meter.

29. The respiratory gas exchange monitor of claim 26, wherein said second sensor is a fluorescence quench oxygen sensor.

30. The respiratory gas exchange monitor of claim 26, wherein said computation unit is configured to compare said respiratory quotient with a reference respiratory quotient to determine a measure of deviation of said respiratory quotient with respect to said reference respiratory quotient.

31. A respiratory gas exchange monitor, comprising:
means for determining a volume of inhaled gases of a subject and a volume of exhaled gases of said subject;
means for determining a concentration of oxygen in said exhaled gases;
means for determining an amount of carbon dioxide produced by said subject and an amount of oxygen consumed by said subject based on said volume of said inhaled gases, said volume of said exhaled gases, and said concentration of oxygen in said exhaled gases; and
means for determining a respiratory quotient of said subject based on a ratio of said amount of carbon dioxide produced and said amount of oxygen consumed.

32. A respiratory gas exchange monitor, said respiratory gas exchange monitor being configured to perform a method comprising:
determining a volume of inhaled gases and a volume of exhaled gases;
determining a speed of sound in said exhaled gases;
determining an amount of carbon dioxide produced and an amount of oxygen consumed based on said volume of said inhaled gases, said volume of said exhaled gases, and said speed of sound in said exhaled gases; and
determining a respiratory quotient based on said amount of carbon dioxide produced and said amount of oxygen consumed.

33. A method of determining a respiratory quotient of a subject, comprising:
determining a volume of inhaled gases of said subject and a volume of exhaled gases of said subject;
determining a mass of carbon dioxide and oxygen in said exhaled gases;
determining a concentration of oxygen in said exhaled gases based on said mass of carbon dioxide and oxygen in said exhaled gases;
determining an amount of carbon dioxide produced by said subject and an amount of oxygen consumed by said subject based on said volume of said inhaled gases, said volume of said exhaled gases, and said concentration of oxygen in said exhaled gases; and
determining a respiratory quotient of said subject based on said amount of carbon dioxide produced and said amount of oxygen consumed.

34. The method of claim 33, wherein determining said mass of carbon dioxide and oxygen in said exhaled gases includes:
determining a mass of said exhaled gases;
determining a mass of nitrogen in said exhaled gases; and
determining said mass of carbon dioxide and oxygen in said exhaled gases based on said mass of said exhaled gases and said mass of nitrogen in said exhaled gases.
